(19) United States
(12) Patent Application Publication
(10) Pub. No.: US 2016/0019688 A1
(43) Pub. Date: Jan. 21, 2016

(54) METHOD AND SYSTEM OF ESTIMATING PRODUCE CHARACTERISTICS

(71) Applicant: University of Georgia Research Foundation, Inc., Athens, GA (US)

(72) Inventors: Changying Li, Athens (CN); Weilin Wang, Athens (CN)

(21) Appl. No.: 14/802,281

(22) Filed: Jul. 17, 2015

Related U.S. Application Data
(60) Provisional application No. 62/026,261, filed on Jul. 18, 2014.

(51) Int. Cl. G06T 7/00; G06K 9/52; H04N 13/02; G01G 19/414; G06T 15/00; G01N 33/02; G01G 9/00; G06T 7/60; H04N 5/225

(52) U.S. Cl. CPC G06T 7/0004; G06T 7/602; G06K 9/52; G06T 7/0075; H04N 13/02; H04N 5/2256; G06T 15/005; G01N 33/025; G01G 9/00; G01G 19/414; H04N 2013/0081

(57) ABSTRACT

Disclosed are various embodiments for a method, system, and apparatus for taking three-dimensional images of produce. The three-dimensional image may be used to estimate the volume and other dimensions of the imaged produce.

### CROSS REFERENCE TO RELATED APPLICATIONS

This application claims priority to and the benefit of U.S. Provisional Application No. 62/026,261, filed on Jul. 18, 2014, which is incorporated by reference herein in its entirety as if set forth below.

### STATEMENT REGARDING FEDERALLY SPONSORED RESEARCH OR DEVELOPMENT

This invention was made with government support under grant no. 2009-51181-06010 awarded by the U.S. Department of Agriculture. The government has certain rights in this invention.

### BACKGROUND

The size of fruits and vegetables is an essential physiological property that can be described by different parameters such as volume, weight, length, and diameter. Size determination is often mandatory to the sorting of many fruits and vegetables for various reasons, such as requirements of processing machine, regulatory rules of sorting standards, and consumer preferences. Size is also an important quantitative factor to evaluate for phenotyping of fruits and vegetables.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is drawing of a three-dimensional produce imager according to various embodiments of the present disclosure.

FIG. 2 is a drawing of a networked environment according to various embodiments of the present disclosure.

FIG. 3 is a flowchart illustrating one example of functionality implemented as portions of an application executed in a computing environment in the networked environment of FIG. 2 according to various embodiments of the present disclosure.

FIG. 4 is a flowchart illustrating one example of functionality implemented as portions of an application executed in a computing environment in the networked environment of FIG. 2 according to various embodiments of the present disclosure.

FIG. 5 is a schematic block diagram that provides one example illustration of a computing environment employed in the networked environment of FIG. 1 according to various embodiments of the present disclosure.

### DETAILED DESCRIPTION

In the following discussion, a general description of the system and its components is provided, followed by a discussion of the operation of the same.

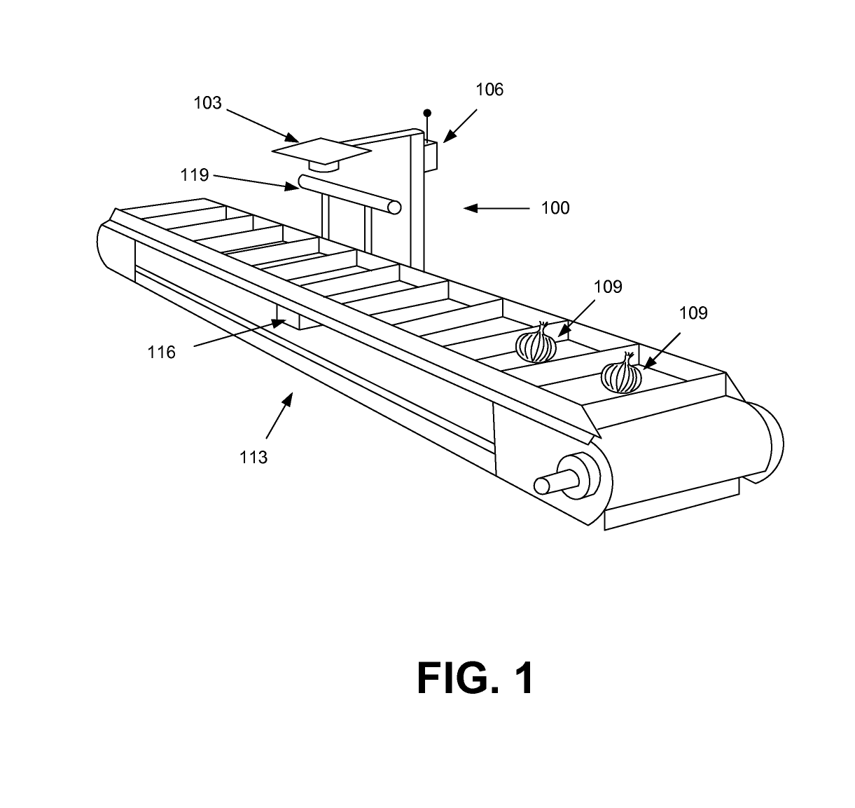

Beginning with FIG. 1, shown is a three-dimensional produce imager 100 according to various embodiments of the present disclosure. The three-dimensional produce imager 100 comprises a Red-Green-Blue-Depth (RGB-D) sensor 103 and a network interface 106. As produce items 109 move along a conveyor belt 113, each of the produce items 109 proceed through the field of view of the RGB-D sensor 103. The RGB-D sensor 103 takes an image of the produce item 109. The image is then sent to a remote computing device via the network interface 106 for further analysis, as will be further described herein.

In some embodiments, the conveyor belt 113 may also include a weighing device 116, such as a scale or other device configured to determine the mass and/or weight of the produce item 109. The weighing device 116 may be positioned below the RGB-D sensor 103 and connected to the three-dimensional produce imaged 100. In such embodiments, the three-dimensional produce imager 100 may record a weight of an individual produce item 109 when the RGB-D sensor 103 takes an image of the produce item 109. In such embodiments, the weight may be sent to the remote computing device via the network interface 106 along with the image taken by the RGB-D sensor 103.

In some embodiments, a light source 119 may also be present. Ambient light, such as sunlight or light from various lighting sources in the environment, may be sufficient for the RGB-D sensor 103 to properly image an individual produce item 109 in some embodiments. However, other embodiments may be located in environments where ambient light is insufficient. Accordingly, some embodiments may include a dedicated light source 119 to provide sufficient light for operation of the RGB-D sensor 103.

The RGB-D sensor 103 includes an RGB color camera, a monochrome infrared camera, and a laser emitter. The laser emitter projects a structured infrared lighting pattern and the monochrome infrared camera captures the imposed pattern shined on a produce item 109. The RGB color camera takes a color photograph of the product item 109 when the produce item 109 within the field of view of the RGB color camera and, therefore, the field of view of the RGB-D sensor 103. The field of view is the viewable spatial area in which the RGB-D sensor 103 can detect and/or generate an image of the produce item 109.

The network interface 106 may include any one or more of a number of network interfaces. For example, the network interface 106 may permit wireless transmissions of network traffic, such as Wi-Fi® or Bluetooth® networking. As another example, the network interface 106 may include a wire or cable to another computing device or networking device. In such embodiments, the network interface 106 may include an Ethernet card, a fiber-optic networking card, a modem, or other such device.

Produce items 109 may include most types of fresh fruits and vegetables, and/or other food stuffs. As an illustrative and non-limiting example, produce items 109 may include onions, squash, zucchini, bell peppers, habanero peppers, jalapeno peppers, garlic cloves, tomatoes, potatoes, coconuts, oranges, apples, plums, plouts, kiwi fruit, lemons, limes, grapefruits, and/or other such items.

In other embodiments of the present disclosure, one or more of the components depicted in FIG. 1 may be positioned in a different location and/or orientation than is depicted in FIG. 1. For example, the RGB-D sensor 103 may be placed to the side of the conveyor belt 113 or underneath the produce items 109 in a manner similar to the positioning of the weighing device 116 as depicted in FIG. 1. As another example, the light source 119 may be positioned more directly overhead or may be positioned underneath the produce items 109 as they move along the conveyor belt 113.

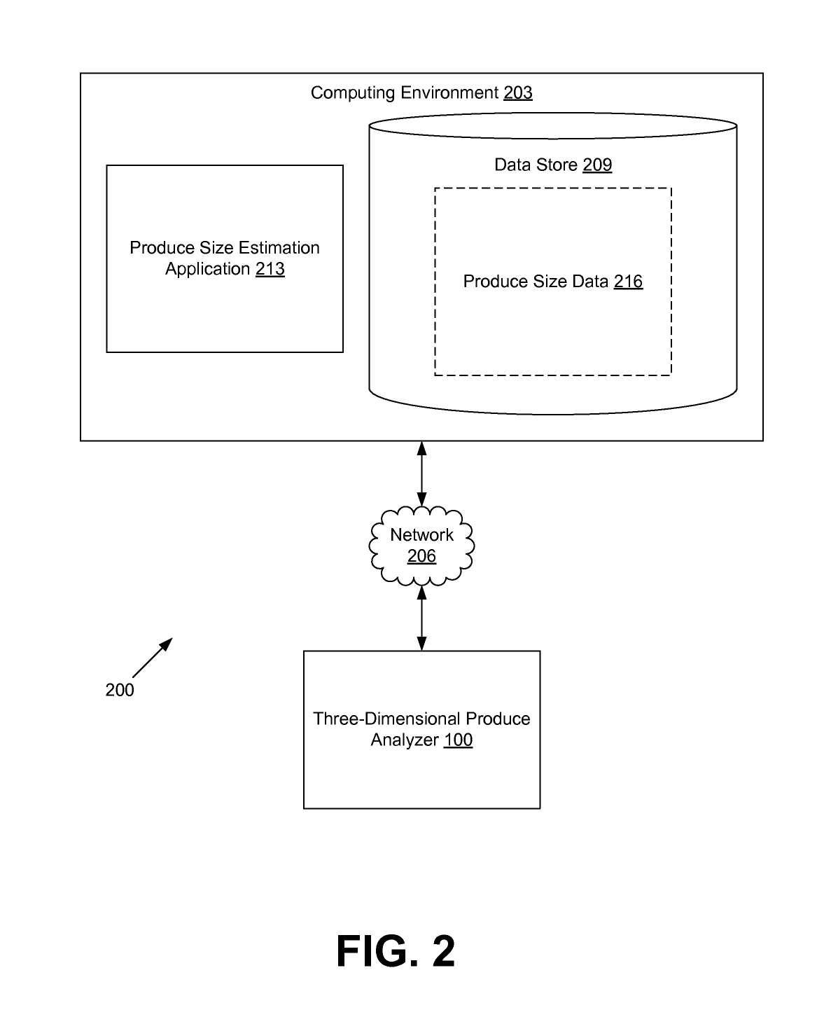

With reference to FIG. 2, shown is a networked environment 200 according to various embodiments. The networked environment 200 includes a computing environment 203 and a three-dimensional produce imager 100, which are in data communication with each other via a network 206. The network 206 includes, for example, the Internet, intranets, extranets, wide area networks (WANs), local area networks (LANs), wired networks, wireless networks, or other suitable networks, etc., or any combination of two or more such networks. For example, such networks may comprise satellite networks, cable networks, Ethernet networks, and other types of networks.

The computing environment 203 may comprise, for example, a server computer or any other system providing computing capability. Alternatively, the computing environment 203 may employ a plurality of computing devices that may be arranged, for example, in one or more server banks, computer banks, or other arrangements. Such computing devices may be located in a single installation or may be distributed among many different geographical locations. For example, the computing environment 203 may include a plurality of computing devices that together may comprise a hosted computing resource, a grid computing resource and/or any other distributed computing arrangement. In some cases, the computing environment 203 may correspond to an elastic computing resource where the allotted capacity of processing, network, storage, or other computing-related resources may vary over time.

Various applications and/or other functionality may be executed in the computing environment 203 according to various embodiments. Also, various data is stored in a data store 209 that is accessible to the computing environment 203. The data store 209 may be representative of a plurality of data stores 209 as can be appreciated. The data stored in the data store 209, for example, is associated with the operation of the various applications and/or functional entities described below.

The components executed on the computing environment 203, for example, include a produce size estimation application 213 and other applications, services, processes, systems, engines, or functionality not discussed in detail herein. The produce size estimation application 213 is executed to estimate the volume of individual produce items 109 (FIG. 1) based at least in part on an image of the produce item 109 provided by the three-dimensional produce analyzer 100.

The data stored in the data store 209 includes, for example, produce size data 216, and potentially other data. The produce size data 216 represents data related to the volume of individual produce items 109. The produce size data 216 may, for example, include the result of a volume calculation or computation of an individual produce item 109 performed by the produce size estimation application 213, an identifier of the individual produce item 109, and/or the image of the individual produce item 109.

Next, a general description of the operation of the various components of the networked environment 200 is provided. To begin, the three-dimensional produce analyzer 100 generates an image of a produce item 109 using an RGB-D sensor 103 (FIG. 1). The image may include a two-dimensional color image of the produce item 109 and/or a depth image of the produce item 109. The three-dimensional produce analyzer 100 then sends the image across the network 206 to the produce size estimation application 213.

The produce size estimation application 213 then calculates the volume of the produce item 109 based at least in part on the image received from the three-dimensional produce analyzer 100. First, the produce size estimation application 213 identifies the major axis for the produce item 109 from the received image. The major axis of the produce item 109 may be calculated using the color image and/or the depth image received from the three-dimensional produce analyzer 100. The size estimation application 213 similarly identifies the minor axis of the produce item 109 using the color image and/or the depth image received from the three-dimensional produce analyzer 100. The size estimation application 213 may also identify the height of the product item 109 for various sections of the produce item 109 using the depth image received from the three-dimensional produce analyzer. The produce size estimation application 213 may then estimate the volume of the produce item 109 based at least in part on the major axis, the minor axis, and the height.

For example, to estimate the volume of a produce item 109, a point cloud image may be converted into a voxel image, in which each pixel is treated as a box rather than a point, forming a 3-D block figure. The total volume of the produce item 109 can be calculated by summing up the volume of all boxes within the region of the point cloud image corresponding to the produce item 109. The volume of each box may be calculated using the equation V=height(mm)×pixel resolution(mm/pixel)×pixel resolution (mm/pixel).

Moreover, the produce size estimation application 213 may receive a weight of the produce item 109 from a weighing device 116 (FIG. 1). The weight of the produce item 109 may be used to estimate the density of the produce item 109. In some embodiments, the density of the produce item 109 may also be used to verify the accuracy of the estimated volume of the produce item 109. For example, if a particular type of produce item 109 has an average density of X, but the estimated volume of the produce item 109 and the weight of the produce item 109 indicates a density that deviates too far from the average density of X, such as density one-and-a-half, two, three, or more times the value of X, the produce size estimation application 213 may determine that one or more of the major axis, minor axis, or height of the produce item 109 was incorrectly measured.

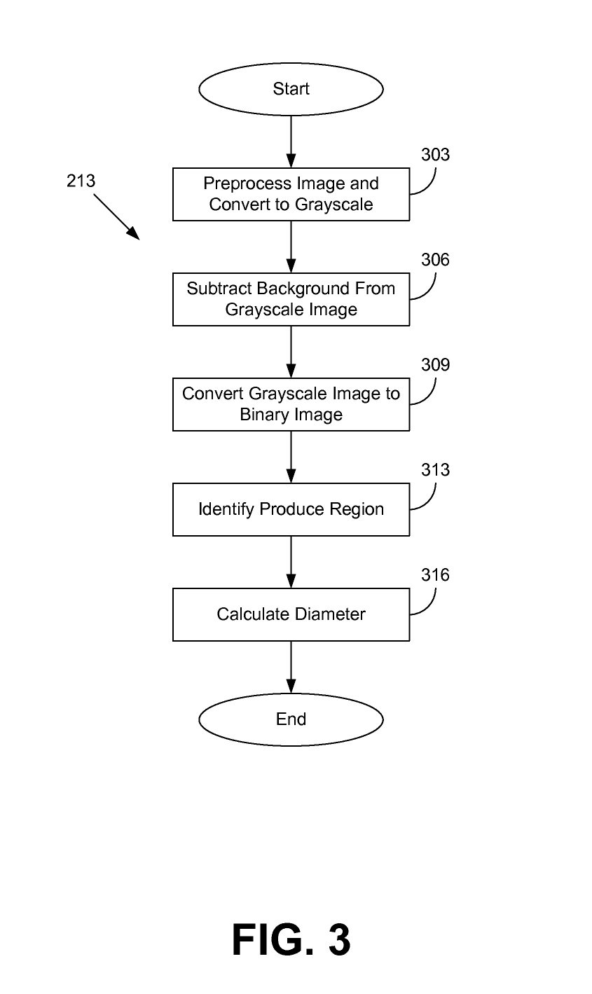

Referring next to FIG. 3, shown is a flowchart that provides one example of the operation of a portion of the produce size estimation application 213 according to various embodiments. It is understood that the flowchart of FIG. 3 provides merely an example of the many different types of functional arrangements that may be employed to implement the operation of the portion of the produce size estimation application 213 as described herein. As an alternative, the flowchart of FIG. 3 may be viewed as depicting an example of elements of a method implemented in the computing environment 203 (FIG. 2) according to one or more embodiments.

Beginning with box 303, the produce size estimation application 213 preprocesses an image of a produce item 109 (FIG. 1) and converts the pre-processed image to grayscale. For example, sub-images may be extracted from the center of color images corresponding to the area of underneath the RGB-D sensor 103 (FIG. 1). The extracted sub-images may then be converted to grayscale.

Proceeding next to box 306, the background of the grayscale image, which may correspond to the conveyor belt 113 (FIG. 1) carrying produce items 109 underneath the RGB-D sensor 103 (FIG. 1) are subtracted from the grayscale images.

Moving on to box 309, the remaining portions of the grayscale image may be converted to a binary image. For example, the produce size estimation application 213 may use Otsu's method, or similar approaches, to convert the remaining portions of the grayscale image to a binary image.

Referring next to box 313, the produce size estimation application 213 identifies a region within the binary image corresponding to the produce item 109. The produce size estimation application 213 may accomplish this by applying morphological close operations to remove internal spots within various regions of the binary image. The region with the largest area in the binary image may then be recognized as the region corresponding to the produce item 109.

Proceeding on to box 316, the produce size estimation application 213 calculates the diameter of the produce item 109. The produce size estimation application 213 calculates a peripheral ellipse that has the same normalized second central moments as the region identified as corresponding to the produce item 109. The major axis of the peripheral ellipse is identified as the major axis of the produce item 109. The maximum diameter of the produce item 109 is computed by multiplying the pixel length of the major axis by the pixel resolution of the binary image and/or original image. Execution subsequently ends.

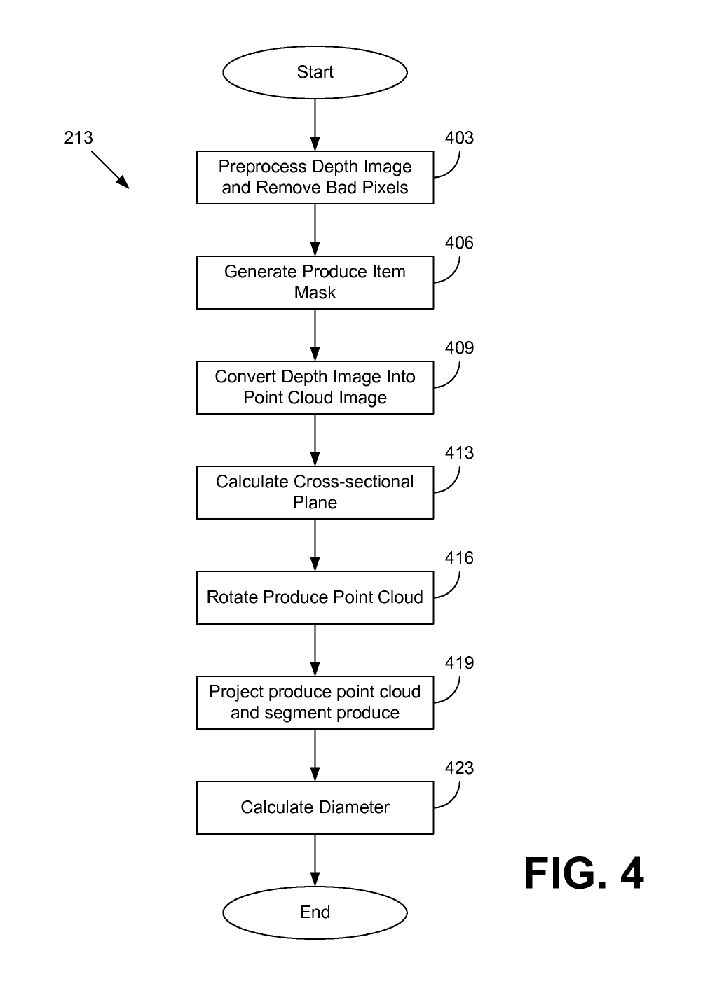

Referring next to FIG. 4, shown is a flowchart that provides one example of the operation of a portion of the produce size estimation application 213 according to various embodiments. It is understood that the flowchart of FIG. 4 provides merely an example of the many different types of functional arrangements that may be employed to implement the operation of the portion of the produce size estimation application 213 as described herein. As an alternative, the flowchart of FIG. 4 may be viewed as depicting an example of elements of a method implemented in the computing environment 203 (FIG. 2) according to one or more embodiments.

Beginning with box 403, the produce size estimation application 213 preprocesses a depth image of a produce item 109 (FIG. 1) generated by an RGB-D sensor 103 (FIG. 1) to remove bad pixels. A center area of the depth image may be extracted. Pixels in dark holes in the depth image may be replaced by the mean pixel value of its nearest valid neighborhood points.

Proceeding next to box 406, the produce size estimation application 213 generates a mask of the produce item 109. The mask may be generated by differentiating the depth image of the background and the depth image of the depth image of the produce item 109. The difference image generated is converted into a binary image after applying a previously defined threshold value, which may be empirically determined for individual types of produce, to remove false foreground objects, such as debris on the conveyor belt 113 (FIG. 1). The binary region with the largest area is recognized as the initial produce mask.

Moving on to box 409, the depth image is converted into a point cloud image of the produce item 109. The background in the depth image is removed by applying the mask generated in box 406. The produce size estimation application 213 may then, for example, subtract the depth value by the mean depth of the scanning stage at each pixel. After conversion, the pixel value of the point could image of the produce item 109 refers to the height of the produce item 109 relative to its position on the conveyor belt 113 (FIG. 1).

Referring next to box 413, a cross-sectional plane for the produce item 109 may be calculated. In some embodiments, the orientation of the produce item 109 is estimated to correct for tilting of the produce item 109. To estimate the orientation of the produce item 109, the edge of the point cloud image is recognized. To recognize the edge, the produce size estimation application 213 may apply the following or similar approaches. First, the edge of the produce item 109 seen by the RGB-D sensor 103 may be represented by the edge of the equatorial cross section of the produce item 109. In such embodiments, E(x, y, z) may denote the edge points of the point cloud image I (E⊂I). To recognize the orientation of the produce item 109, a principal component analysis (PCA) may be performed on E. In the PCA transformation, the covariance matrix C of E may be computed: C=UΛU^{T}, where Λ may be the diagonal matrix of the eigenvalues of C. U may contain the three eigenvectors: (u₁, u₂, u₃), which may represent three orthonormal principal axes of the data set. The first two principal axes (u₁, u₂) may be identified as a cross sectional plane that has the maximum projection area passing the edge points E. This plane may represent approximately the sectional plane crossing the equator of the produce item 109. The third axis u₃, orthonormal to u₁ and u₂, may represent the normal vector of the fitted plane, which may represent an estimation of the overall orientation of the produce item 109.

Proceeding on to box 416, the produce size estimation application 213 rotates the point cloud image. The produce size estimation application 213 may calculate the angles (α,θ) between the plane and the X, Y axes using the normal vector u₃. The 3-D geometrical transformation matrixes Rₓ and R_{y} may then be calculated using the calculated angles (α,θ). A rigid transformation may also be performed to rotate the point cloud image along the X and/or Y axis (I′=RₓR_{y}I), in order to maximize the projection area parallel to the X-Y plane.

Moving next to box 419, the transformed point cloud image may be projected into the X-Y plane to generate a binary map for the produce item 109.

Finally, at box 423, the produce size estimation application 213 calculates the diameter of the produce item 109. The produce size estimation application 213 calculates a peripheral ellipse of a region of the binary map identified as corresponding to the produce item 109. The major axis of the peripheral ellipse is identified as the major axis of the produce item 109. The maximum diameter of the produce item 109 is computed by multiplying the pixel length of the major axis by the pixel resolution of the binary image and/or original image. Execution subsequently ends.

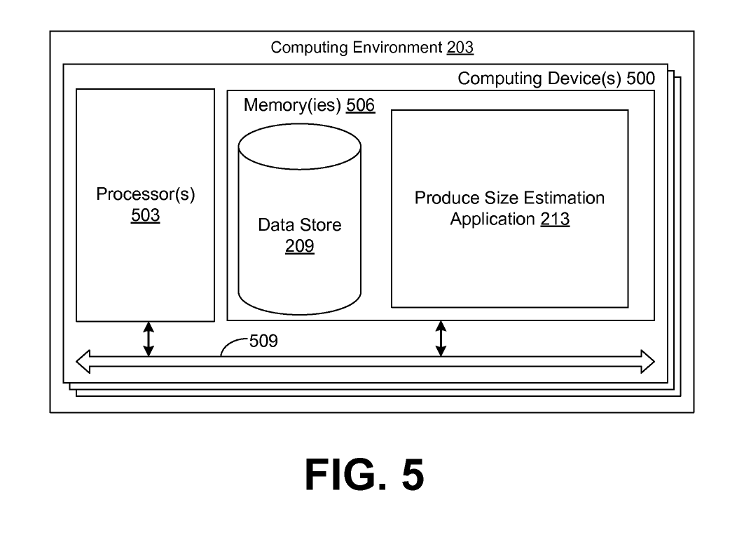

With reference to FIG. 5, shown is a schematic block diagram of the computing environment 203 according to an embodiment of the present disclosure. The computing environment 203 includes one or more computing devices 500. Each computing device 500 includes at least one processor circuit, for example, having a processor 503 and a memory 506, both of which are coupled to a local interface 509. To this end, each computing device 500 may comprise, for example, at least one server computer or like device. The local interface 509 may comprise, for example, a data bus with an accompanying address/control bus or other bus structure as can be appreciated.

Stored in the memory 506 are both data and several components that are executable by the processor 503. In particular, stored in the memory 506 and executable by the processor 503 are the produce size estimation application 213, and potentially other applications. Also stored in the memory 506 may be a data store 209 and other data. In addition, an operating system may be stored in the memory 506 and executable by the processor 503.

It is understood that there may be other applications that are stored in the memory 506 and are executable by the processor 503 as can be appreciated. Where any component discussed herein is implemented in the form of software, any one of a number of programming languages may be employed such as, for example, LabVIEW, C, C++, C#, Objective C, Java®, JavaScript®, Perl, PHP, Visual Basic®, Python®, Ruby, Flash®, or other programming languages.

A number of software components are stored in the memory 506 and are executable by the processor 503. In this respect, the term “executable” means a program file that is in a form that can ultimately be run by the processor 503. Examples of executable programs may be, for example, a compiled program that can be translated into machine code in a format that can be loaded into a random access portion of the memory 506 and run by the processor 503, source code that may be expressed in proper format such as object code that is capable of being loaded into a random access portion of the memory 506 and executed by the processor 503, or source code that may be interpreted by another executable program to generate instructions in a random access portion of the memory 506 to be executed by the processor 503, etc. An executable program may be stored in any portion or component of the memory 506 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 506 is defined herein as including both volatile and nonvolatile memory and data storage components. Volatile components are those that do not retain data values upon loss of power. Nonvolatile components are those that retain data upon a loss of power. Thus, the memory 506 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 503 may represent multiple processors 503 and/or multiple processor cores and the memory 506 may represent multiple memories 506 that operate in parallel processing circuits, respectively. In such a case, the local interface 509 may be an appropriate network that facilitates communication between any two of the multiple processors 503, between any processor 503 and any of the memories 506, or between any two of the memories 506, etc. The local interface 509 may comprise additional systems designed to coordinate this communication, including, for example, performing load balancing. The processor 503 may be of electrical or of some other available construction.

Although the produce size estimation application 213, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts of FIGS. 3 and 4 show the functionality and operation of an implementation of portions of the produce size estimation application 213. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 503 in a computer system or other system. The machine code may be converted from the source code, etc. If embodied in hardware, each block may represent a circuit or a number of interconnected circuits to implement the specified logical function(s).

Although the flowcharts of FIGS. 3 and 4 show a specific order of execution, it is understood that the order of execution may differ from that which is depicted. For example, the order of execution of two or more blocks may be scrambled relative to the order shown. Also, two or more blocks shown in succession in FIGS. 3 and 4 may be executed concurrently or with partial concurrence. Further, in some embodiments, one or more of the blocks shown in FIGS. 3 and 4 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the produce size estimation application 213, that comprises software or code can be embodied in any non-transitory computer-readable medium for use by or in connection with an instruction execution system such as, for example, a processor 503 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the produce size estimation application 213, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 500, or in multiple computing devices in the same computing environment 203. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A system, comprising:
a computing device comprising a processor and a memory; and
an application executed in the at least one computing device, the application comprising a set of instructions stored in the memory of the computing device that, when executed by the processor of the computing device, cause the computing device to at least:
convert a depth image of a produce item into a point cloud image; and
estimate a diameter of the produce item based at least in part on the point cloud image.

2. The system of claim 1, wherein the application further comprises instructions stored in the memory of the computing device that, when executed by the processor of the computing device, causes the computing device to at least calculate a volume of the produce item based at least in part on the estimated diameter of the produce item.

3. The system of claim 1, further comprising a Red-Green-Blue-Depth (RGB-D) sensor configured to:
generate the depth image of the produce item; and
send the depth image of the produce item to the computing device.

4. The system of claim 3, further comprising a conveyor belt positioned to move the produce item through a field of view of the RGB-D sensor.

5. The system of claim 3, further comprising a light source positioned to illuminate the produce item when the produce item is positioned within a field of view of the RGB-D sensor.

6. The system of claim 3, wherein the RGB-D sensor is positioned above the produce item.

7. The system of claim 3, wherein the RGB-D sensor is positioned below the produce item.

8. The system of claim 1, further comprising a weighing device configured to:
measure a weight of the produce item; and
send the weight of the produce item to the computing device.

9. The system of claim 8, wherein the application further comprises instructions stored in the memory of the computing device that, when executed by the processor of the computing device, causes the computing device to at least calculate a density of the produce item based at least in part on the weight of the produce item.

10. The system of claim 8, wherein the weighing device comprises a scale.

11. The system of claim 1, wherein the produce item comprises an onion.

12. A non-transitory computer-readable medium comprising a program that, when executed by a processor of a computing device, causes the computing device to at least:
convert a depth image of a produce item into a point cloud image;
estimate a volume of the produce item based at least in part on the point could image; and
estimate a diameter of the produce item based at least in part on the point could image.

13. The non-transitory computer-readable medium of claim 12, wherein the program, when executed by the processor, further causes the computing device to at least compute a weight of the produce item based at least in part on a measurement provided by a weighing device.

14. The non-transitory computer-readable medium of claim 13, wherein the program, when executed by the processor, further causes the computing device to at least estimate a density of the produce item based at least in part on the estimated volume and the computed weight of the produce item.

15. The non-transitory computer-readable medium of claim 12, wherein the depth image is received from a Red-Green-Blue-Depth (RGB-D) sensor configured to:
generate the depth image of the produce item; and
send the depth image of the produce item to the computing device.

16. A computer-implemented method, comprising:
converting a depth image of a produce item into a point cloud image;
estimating a diameter of the produce item based at least in part on the point cloud image; and
estimating a volume of the produce item based at least in part on the estimated diameter.

17. The computer-implemented method of claim 16, further comprising receiving the depth image of the produce item from a Red-Green-Blue-Depth (RGB-D) sensor, wherein the RGB-D sensor generates the depth image.

18. The computer-implemented method of claim 16, further comprising receiving a weight of the produce item from a weighing device.

19. The computer-implemented method of claim 18, further comprising estimating a density of the produce item based at least in part on the volume of the produce item and the weight of the produce item.

20. The computer-implemented method of claim 16, wherein converting the depth image of the produce item into a point cloud image further comprises removing a pixel from the depth image.
